(19) United States
(12) Patent Application Publication
(10) Pub. No.: US 2020/0365233 A1
(43) Pub. Date: Nov. 19, 2020

(54) METHOD AND ELECTRONIC DEVICE FOR HANDLING ENZYME FUNCTION THROUGH AT LEAST ONE CHANGE AT MULTIPLE SITES IN ENZYME

(71) Applicant: Samsung Electronics Co., Ltd., Suwon-si (KR)

(72) Inventors: Garima AGARWAL, Bangalore (IN); Tadi Venkata SIVA KUMAR, Bangalore (IN); Rajasekhara Reddy DUVVURU MUNI, Bangalore (IN); Taeyong Kim, Daejeon (KR)

(21) Appl. No.: 16/745,013

(22) Filed: Jan. 16, 2020

(30) Foreign Application Priority Data
May 15, 2019 (IN) 201941019327
Sep. 19, 2019 (KR) 10-2019-0115486

(51) Int. Cl. G16B 20/30

(52) U.S. Cl. CPC G16B 20/30

(57) ABSTRACT

Accordingly, embodiments herein disclose a method for handling an enzyme function through at least one change at multiple sites in an enzyme. The method includes detecting, by an electronic device, one or more functionally linked site pairs from the multiple sites in the enzyme. Further, the method includes determining, by the electronic device, at least one criticality assessment function for the one or more functionally linked site pairs. Further, the method includes prioritizing, by the electronic device, the functionally linked site pairs based on at least one of the criticality assessment function, a functional linkage strength, and a spatial linkage metric. Further, the method includes identifying, by the electronic device, the multiple sites from the prioritized functionally linked site pairs as optimum sites for introducing a change to handle the enzyme function.

### CROSS REFERENCE TO RELATED APPLICATION

This application claims the benefit of and priority to Indian Patent Application 201941019327, filed on May 15, 2019, and Korean Patent Application No.10-2019-0115486, filed on Sep. 19, 2019, the disclosures of which are incorporated by reference herein in their entireties.

### BACKGROUND

### 1. Field

The present disclosure relates to enzyme engineering, and more specifically to a method and system for handling an enzyme function through at least one change at multiple sites in an enzyme.

### 2. Description of the Related Art

Microbial industrial scale production/degradation of molecules of interest requires efficient enzymes tuned to the desired conditions and biochemical reaction. In order to enable cost-effective production/degradation at the industrial scale, enzymes often need to be engineered to enhance their functional properties such as activity, specificity, stability and affinity. Enzyme engineering requires systematic exploration and evaluation of changes often at more than one site. Evaluating these changes experimentally is a time-consuming, labor intensive process with a low success rate and is often infeasible for large systems. Insilco methods provide a cost effective alternative to identify/shortlist sites for engineering for efficient enzyme design with reduced search space.

Insilico identification of sites for engineering the desired functional change is challenging. While identifying and modifying sites of functional importance needs to be prioritized, changes to sites critical for structural and functional integrity need to be minimized. Further, a site could be functionally dependant on other sites. Hence changes at these sites are not mutually exclusive. In order to increase the success rate and minimize negative impacts of the enzyme engineering process, the selected sites should have minimal functional linkage with the rest of the enzyme.

Limited computational methods are available to predict changes at multiple sites to enhance functional properties.

One method to predict multiple sites for engineering is Hotspot (Bendl et al, 2016 Nucleic Acids Research, 44: W479-W487). The selection of sites is based on degree of mutability and the correlation of sites. The presence of correlation, however, is not always indicative of functional relevance.

Thus, it is desirable to consider/address the above mentioned factors/challenges or other shortcomings and provide a useful alternative.

### SUMMARY

An object of embodiments herein is to provide a method and an electronic device for handling an enzyme function through at least one change at multiple sites in an enzyme.

Another object of embodiments herein is to detect, by an electronic device, at least one functionally linked site pair from the multiple sites in the enzyme.

Another object of embodiments herein is to determine, by the electronic device, at least one criticality assessment function for one or more functional sites/functionally linked sites.

Another object of embodiments herein is to prioritize, by the electronic device, one or more functionally linked site pairs based on the at least one of the criticality assessment function, a functional linkage strength, and a structural linkage metric of the functionally linked site pairs.

Another object of embodiments herein is identify, by the electronic device, the multiple sites from the at least one prioritized functionally linked site pairs.

Another object of embodiments herein is to handle, by the electronic device, the enzyme function of the object through the at least one change at multiple sites based on the at least one prioritized functionally linked site pairs.

Another object of embodiments herein is to enable, by the electronic device, enhancement to the enzyme function in the at least one change at multiple sites in the prioritized functionally linked site pairs.

Another object of embodiments herein is to reduce, by the electronic device, the number of functionally linked site pairs to be evaluated for user-desired changes in the enzyme function of the object.

Another object of embodiments herein is to reduce, by the electronic device, the number of functionally linked site pairs to be evaluated through assessment of linkage with at the user-defined site for user-desired changes in the enzyme function of the object.

Another object of embodiments herein is to propose, by the electronic device, the at least one change at multiple sites by combining the at least one functionally linked pairs at a user defined site.

Another object of embodiments herein is compute, by the electronic device, the criticality assessment function for every non-functional site, wherein the non-functional site is ranked based on criticality score and enhances enzyme function through changes in enzyme stability through at least one change at multiple sites.

Additional aspects will be set forth in part in the description which follows and, in part, will be apparent from the description, or may be learned by practice of the presented embodiments of the disclosure.

Accordingly, embodiments herein disclose a method for handling an enzyme function through at least one change at multiple sites in an enzyme. The method includes detecting, by an electronic device, one or more functionally linked site pairs from the multiple sites in the enzyme. Further, the method includes determining, by the electronic device, at least one criticality assessment function for the one or more functionally linked site pairs. Further, the method includes prioritizing, by the electronic device, the one or more functionally linked site pairs based on the at least one of the criticality assessment function, a functional linkage strength, and a spatial linkage metric. Further, the method includes identifying, by the electronic device, the multiple sites from the one or more prioritized functionally linked site pairs.

In an embodiment, the one or more functionally linked site pairs may be detected by determining at least one functional site, detecting a linkage of the at least one functional site to another site (i.e., linked site), and detecting an information flow in the linkage of the at least one functional site to the other site.

In an embodiment, a functional site is determined by identifying the at least one functional site that defines the ligand binding pocket of the enzyme.

In an embodiment, determining the functional site includes identifying the at least one functional site that defines a ligand binding pocket of the enzyme, and identifying the at least one functional site that defines a sequence context of a ligand binding site identified in the binding pocket of the enzyme.

In an embodiment, detecting the linkage of the at least one functional sites to other sites includes identifying the at least one site linked to functional sites using an evolutionary constraint parameter, and removing one or more functional site pairs with no information flow between the site pairs.

In an embodiment, the information flow in the linkage of the at least one functional sites to other site is assessed for evidence of functional communication through a network of spatial links.

In an embodiment, the criticality assessment function for the linked site and the at least one functionally linked site pairs are determined based on a protein feature parameter at a specified site and a neighborhood feature parameter.

In an embodiment, the protein feature parameter at the specified site is computed based on at least one of a structural parameter, an extent of evolutionary constraint, and at least one physicochemical property of an amino acid at the site.

In an embodiment, the neighborhood feature parameter is computed based on at least one of a contact score and a co-dependency index value.

In an embodiment, the method includes enabling enhancement to the enzyme function through at least one change at multiple sites in the prioritized functionally linked site pairs.

In an embodiment, the method further includes proposing at least one change at multiple sites by combining the at least one functionally linked pairs at a user defined site.

In an embodiment, the criticality assessment function is computed for every non-functional site, wherein the non-functional site is ranked based on a criticality score and enhances enzyme function through changes in enzyme stability through at least one change at multiple sites.

In an embodiment, the multiple identified sites include a block of multiple sites which is collectively handled for the enzyme function.

Accordingly, embodiments herein disclose an electronic device for handling an enzyme function through at least one change at multiple sites in an enzyme. The electronic device includes a processor coupled with a memory. The processor is configured to detect one or more functionally linked site pairs from the multiple sites in the enzyme. The processor is configured to determine one or more criticality assessment functions for the one or more functionally linked site pairs. The processor is configured to prioritize the one or more functionally linked site pairs based on at least one of the criticality assessment function, a functional linkage strength, and a spatial linkage metric. The processor enzyme function handler is configured to identify the multiple sites from the one or more prioritized functionally linked site pairs as optimum sites for introducing a change to handle the enzyme function.

In an embodiment, the electronic device is used to reduce the number of functionally linked site pairs to be evaluated for user-desired changes in the enzyme function of the object.

In an embodiment, the electronic device is used to reduce the number of functionally linked site pairs to be evaluated through assessment of linkage with the user-defined site for user-desired changes in the enzyme function of the object.

In an embodiment, the multiple identified sites are block of multiple sites which is collectively handled for the enzyme function.

In an embodiment, the functional linkage strength is obtained from a value for at least one functionally linked pair, wherein the value is obtained from mutual information score across the at least one functionally linked pair and a number of functionally linked pairs.

In an embodiment, the spatial linkage metric is inversely proportional to a number of edges in a path connecting functional site and the functionally linked site pairs.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The above and other aspects, features, and advantages of certain embodiments of the disclosure will be more apparent from the following description taken in conjunction with the accompanying drawings, in which:

This method is illustrated in the accompanying drawings, throughout which like reference letters indicate corresponding parts in the various figures. The embodiments herein will be better understood from the following description with reference to the drawings, in which:

FIG. 1 is a block diagram of an electronic device for handling an enzyme function of an object through at least one change at multiple sites in an enzyme, according to embodiments as disclosed herein;

FIG. 2 is a block diagram of a processor included in the electronic device for handling the enzyme function of the object through at least one change at multiple sites in the enzyme, according to embodiments as disclosed herein;

FIG. 3 is a flow chart illustrating a method for handling the enzyme function of the object through at least one change at multiple sites in the enzyme, according to embodiments as disclosed herein;

FIG. 4A depicts a detection of functional sites, according to embodiments as disclosed herein;

FIG. 4B depicts a detection of linkage with other sites, according to embodiments as disclosed herein;

FIG. 4C depicts a detection of information flow, according to embodiments as disclosed herein;

FIG. 5A depicts definition of protein features at the site of interest, according to embodiments as disclosed herein;

FIG. 5B depicts definition of properties determined by neighbourhood at the site of interest, according to te embodiments as disclosed herein;

FIG. 6A illustrate percentile ranking for the experimentally validated site pairs leading to affinity change, according to embodiments as disclosed herein;

FIG. 6B illustrates the percentage reduction in search space for function handling, according to the embodiments as disclosed herein; and

FIG. 7 is a graph illustrating detection of critical sites for enzyme stability, according to embodiments as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

As is traditional in the field, embodiments may be described and illustrated in terms of blocks which carry out a described function or functions. These blocks, which may be referred to herein as units or modules or the like, are physically implemented by analog or digital circuits such as logic gates, integrated circuits, microprocessors, microcontrollers, memory circuits, passive electronic components, active electronic components, optical components, hardwired circuits, or the like, and may optionally be driven by firmware and software. The circuits may, for example, be embodied in one or more semiconductor chips, or on substrate supports such as printed circuit boards and the like. The circuits constituting a block may be implemented by dedicated hardware, or by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the invention. Likewise, the blocks of the embodiments may be physically combined into more complex blocks without departing from the scope of the invention

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

Accordingly, embodiments herein provide a method for handling an enzyme function through at least one change at multiple sites in an enzyme. The method includes detecting, by an electronic device, one or more functionally linked site pairs from the multiple sites in the enzyme. Further, the method includes determining, by the electronic device, at least one criticality assessment function for the one or more functionally linked site pairs. Further, the method includes prioritizing, by the electronic device, the one or more functionally linked site pairs based on the at least one of the criticality assessment function, a functional linkage strength, and a spatial linkage metric. Further, the method includes identifying, by the electronic device, the multiple sites from the at least one prioritized functionally linked site pairs as optimum sites for introducing a change to handle the enzyme function.

Unlike conventional methods and systems, the methods herein can be used to handle (e.g., enhance) enzyme function through rational multi-site engineering without external dependencies. The methods can be used to identify sites of functional relevance based on the linkage to known functional sites. The selected site combinations are scored and ranked based on various structural, functional and evolutionary features guided by (i) a site and its neighborhood and (ii) site criticality in an effective manner. In the methods, the prediction of sites can be used to increase enzyme affinity.

The methods can be used to predict sites which can be simultaneously changed to enhance enzyme functional properties.

The methods can be used to reduce the turnaround time in protein engineering and cost minimization in industrial scale degradation of pollutants and synthetic molecules. The methods can be applied to any industry such as pharma, food, etc., involved in microbial-based synthesis/degradation of molecules of interest. The changes at the sites of the enzyme, predicted using the methods will help enhance the enzyme function.

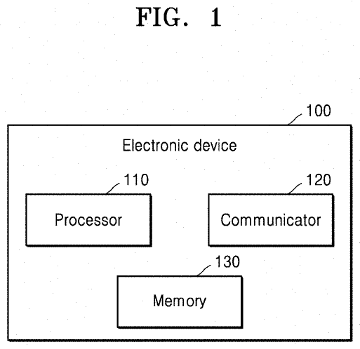

Referring now to the drawings, and more particularly to FIGS. 1 through 7, there are shown certain embodiments.

FIG. 1 is a block diagram of an electronic device 100 for handling an enzyme function of an object through at least one change at multiple sites in an enzyme, according to an embodiment. The electronic device 100 can be, for example, but is not limited to, a smart phone, a laptop, a desktop, a server, a smart watch or the like. The object can be, for example, but is not limited to, a drug item, a bioactive compound, a food item, a pharma item or the like. The electronic device 100 includes a processor 110, a communicator 120, and a memory 130.

In an embodiment, the processor 110 is configured to detect at least one functionally linked site pair from the multiple sites in the enzyme. In an embodiment, the at least one functionally linked site pair is detected by determining at least one functional site, detecting a linkage of the at least one functional site to another site (i.e., functionally linked site pair(s)), and detecting an information flow in the linked sites. The functional site can be a user-defined, sequence-feature based functional site.

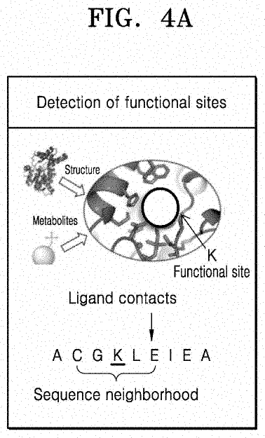

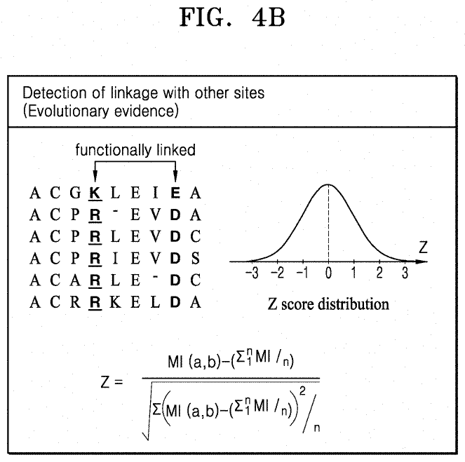

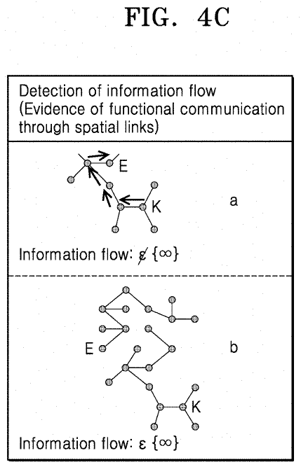

In an embodiment, the at least one functional site is determined by identifying at least one functional site that defines a ligand binding pocket of the enzyme, and further extending it to sites in the sequence neighborhood of the sites identified in the binding pocket of the enzyme. In an example, as shown in the FIG. 4A, K in the enzyme is a functional site.

In an embodiment, the linkage of the at least one functional site to another site is detected by identifying at least one site linked to a functional site using an evolutionary constraint parameter, and removing at least one functional site pair with no information flow therebetween.

In an example, as shown in the FIG. 4B, the linkage with other sites may be identified using mutual information based computations. These computations use sequence alignment and identify the sites that co-vary. Statistically significant sites could be extracted through a thresholding of a vstatistic parameter such as a zscore. In an embodiment, the functional linkage strength could be the zscore value for a linked pair (equation 1).

$$z = \frac{MI ( a , b ) - ( {Σ}_{1}^{n} MI \text{/} n )}{\sqrt{{Σ ( MI ( a , b ) - ( {Σ}_{1}^{n} MI \text{/} n ) )}^{2} \text{/} n}} \qquad ( 1 )$$

where, MI (a,b) is the mutual information score for a pair a and b, and
MI is mutual information score across any functional pair and n is the number of functional pairs.✓

In an embodiment, the information flow in the linkage of the at least one functional site to another site is assessed for evidence of functional communication through a network of spatial links.

In an example, as shown in the FIG. 4C, the information flow in the linkage of the at least one functional site to another site is detected. The enzyme is represented as a graph with nodes depicting sites and edges representing a non-bonded interaction. Two sites linked functionally will interact and are expected to connect through a direct edge/indirect edge. In an example, the path between the E and K is available in the notation “a” of FIG. 4C and is selected. The path between the E and K is not available in the notation “b” of the FIG. 4C and these sites are discarded.

Further, the processor 110 is configured to determine at least one criticality assessment function for the one or more functionally linked site pairs. In an embodiment, the criticality assessment function for at least one functionally linked site pair is determined based on a protein feature parameter at a specified site and a neighborhood feature parameter.

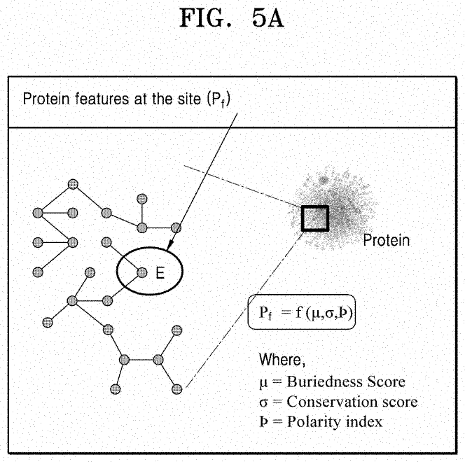

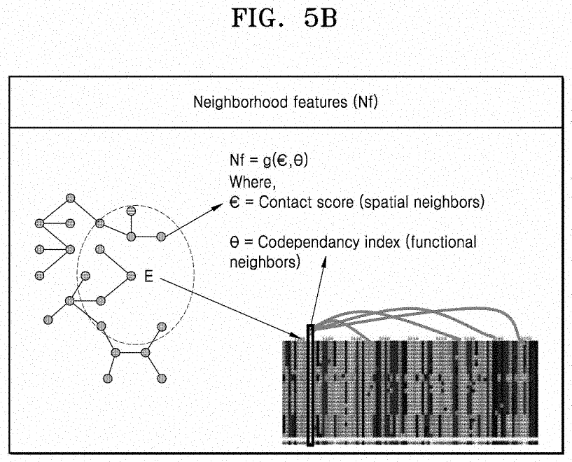

In an embodiment, as shown in the FIG. 5A, the protein feature parameter at the specified site is computed based on at least one of a structural parameter (e.g., buriedness score or the like), an extent of evolutionary constraint (e.g., conservation score or the like), and at least one physicochemical property of an amino acid at the site (e.g., polarity index or the like). The protein feature parameter is determined based on the equation (2)

Pf=f(μ,σ, [symbol] ) (2)

where μ=Buriedness score, σ=Conservation score, [symbol] =Polarity index.

The polarity index is associated with the amino acid at the site, the conservation score is related to the extent of amino acid similarity in related sequences at the site (the conservation score may be computed considering phylogenetic relationship among the sequences in the alignment), and the Buriedness score is related to positioning of the site in the enzyme structure.

In an embodiment, as shown in the FIG. 5B, the neighborhood feature parameter is computed based on at least one of a contact score and a co-dependency index value. The neighborhood feature parameter is represented through the equation (3).

Nf=g(€,⊖) (3)
where, €=Contact score (derived from spatial neighbors), ⊖=Codependency index (derived from functional neighbors)

In an embodiment, the contact score is the degree of a node when the enzyme structure is represented in the form of a graph with nodes defined by sites and edges by the non-bonded interactions.

In an embodiment, the codependency index is the degree of the node when the enzyme structure is represented in the form of a graph with nodes defined by sites and edges by the functional linkage between the sites. In an embodiment, further, the criticality assessment function for the protein feature parameter at the specified site is determined based on the equation (4).

$$Criticality score for site i ( {C}_{i} ) \text{:} \frac{{aP}_{f} + {bN}_{f}}{a + b} \qquad ( 4 )$$

where, a and b are weight values of the features computed for site and site neighbourhood respectively.

Further, the processor 110 is configured to prioritize the functionally linked site pairs based on at least one of the criticality assessment function (Equation 4), the functional linkage strength (as indicated in Equation 1), and a spatial linkage metric.

In an example, the spatial linkage metric is inversely proportional to the number of edges in the path connecting functional site ‘a’ and functionally linked site ‘b’.

In an embodiment, the prioritization may be performed by applying at least one mathematical function on the parameters. The function can be a weighted prioritization, a weighted average, a geometric mean, an arithmetic mean or a mathematical model.

Further, the processor 110 is configured to handle the enzyme function of the object through the at least one change at multiple sites based on at least one of the prioritized functionally linked site pairs. Further, the processor 110 is configured to enable enhancement to the enzyme function in the at least one change at multiple sites in the prioritized functionally linked site pairs.

Further, the processor 110 is configured to identify the multiple sites from the at least one prioritized functionally linked site pairs.

Further, the processor 110 is configured to reduce the number of functionally linked site pairs to be evaluated for user-desired changes in the enzyme function of the object. Further, the processor 110 is configured to reduce the number of functionally linked site pairs to be evaluated through the assessment of linkage with the user-defined site for user-desired changes in the enzyme function of the object.

Further, the processor 110 can be used to enhance enzyme function through changes in enzyme stability based on criticality score parameters of the one or more sites. The functional enhancement could be obtained through multiple ways which includes stability. Further, the processor 110 is configured to propose the at least one change at multiple sites by combining functionally linked pairs at a user defined site.

The processor 110 is configured to execute instructions stored in the memory 130 and to perform various processes. The communicator 120 is configured for communicating internally between internal hardware components and with external devices, e.g., via one or more networks. The communicator 120 is configured for communicating with the processor 110 to handle the enzyme function of the object through at least one change at multiple sites.

The memory 130 also stores instructions to be executed by the processor 110. The memory 130 may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory 130 may, in some examples, be considered a non-transitory storage medium. The term “non-transitory” may indicate that the storage medium is not embodied in a carrier wave or a propagated signal. However, the term “non-transitory” should not be interpreted that the memory 130 is non-movable. In some examples, the memory 130 can be configured to store larger amounts of information than the memory. In certain examples, a non-transitory storage medium may store data that can, over time, change (e.g., in Random Access Memory (RAM) or cache).

Although the FIG. 1 shows various hardware components of the electronic device 100, it is to be understood that other embodiments are not limited thereon. In other embodiments, the electronic device 100 may include fewer or more components. Further, the labels or names of the components are used only for illustrative purpose and do not limit the scope of the invention. One or more components can be combined together to perform a same or substantially similar function to handle the enzyme function of the object through at least one change at multiple sites.

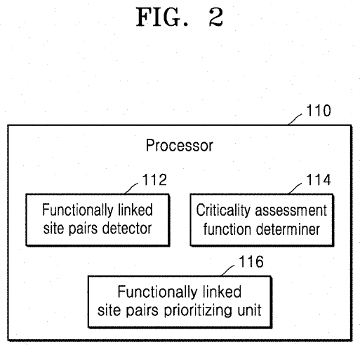

FIG. 2 is a block diagram of the processor 110, according to the embodiments as disclosed herein. In an embodiment, the processor 110 includes a functionally linked site pairs detector 112, a criticality assessment function determiner 114 and a functionally linked site pairs prioritizing unit 116.

The functionally linked site pairs detector 112 is configured to identify at least one functional site that defines the ligand binding pocket of the enzyme and/or sequence neighborhood of pocket residues. The functionally linked site pairs detector 112 is configured to identify at least one functional site that defines the sequence context of the ligand binding site identified in the binding pocket of the enzyme. Further, the functionally linked site pairs detector 112 is configured to determine at least one functional site. Further, the functionally linked site pairs detector 112 is configured to detect the linkage of at least one functional site to another site. The functionally linked site pairs detector 112 is configured to detect an information flow in the linked site(s).

The criticality assessment function determiner 114 is configured to determine at least one criticality assessment function for at least one functionally linked site pair. The functionally linked site pairs prioritizing unit 116 is configured to prioritize the functionally linked site pairs based on at least one of the criticality assessment function, the functional linkage strength, and the spatial linkage metric.

Although FIG. 2 shows various hardware components of the processor 110, it is to be understood that other embodiments are not limited thereon. In other embodiments, the processor 110 may include fewer or more components. Further, the labels or names of the components are used only for illustrative purpose and do not limit the scope of the invention. One or more components can be combined together to perform a same or substantially similar function to handle the enzyme function of the object through at least one change at multiple sites.

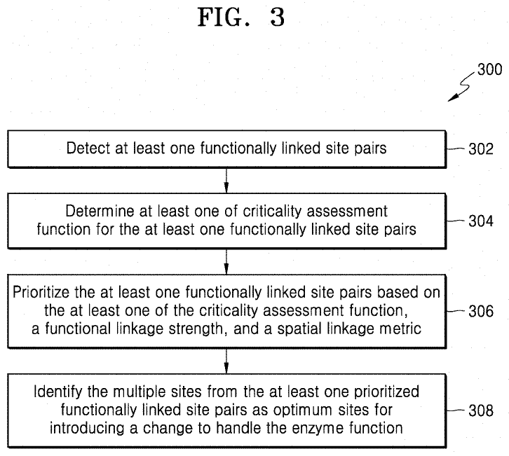

FIG. 3 is a flow chart 300 illustrating a method for handling the enzyme function of the object through at least one change at multiple sites in the enzyme, according to an embodiment. The steps (302-308) may be performed by the processor 110. At step 302, the method includes detecting one or more functionally linked site pairs from the multiple sites in the enzyme. At step 304, the method includes determining at least one criticality assessment function for the one or more functionally linked site pairs. At step 306, the method includes prioritizing the one or more functionally linked site pairs based on at least one of the criticality assessment function, the strength of the functional structural linkage, the degree of the structural linkage, and the spatial connect parameter of the functionally linked site pairs. At step 308, the method includes identifying multiple sites from the prioritized functionally linked site pairs as optimum sites for introducing the change to handle the enzyme function.

The various actions, acts, blocks, steps, or the like in the flow diagram 300 may be performed in the order presented, in a different order or simultaneously. Further, in some embodiments, some of the actions, acts, blocks, steps, or the like may be omitted, added, modified, skipped, or the like without departing from the scope of the invention.

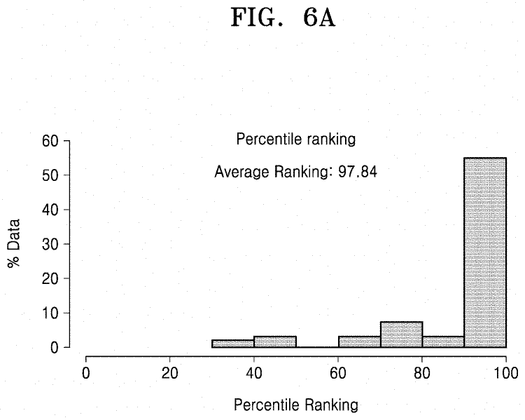

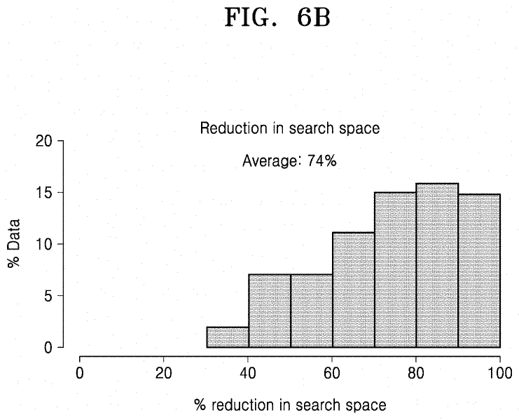

FIG. 6A illustrates percentile ranking for experimentally validated site pairs leading to affinity change, according to the embodiments as disclosed herein. FIG. 6B illustrates the percentage reduction in search space for function handling based on the proposed method, according to the embodiments as disclosed herein.

In an example, in FIG. 6A, in the dataset used, an experimentally validated mutation pair lies in top 2.5%. FIG. 6B illustrates reduction in search space. In the dataset used, the method reduced the search space by 74% on average.

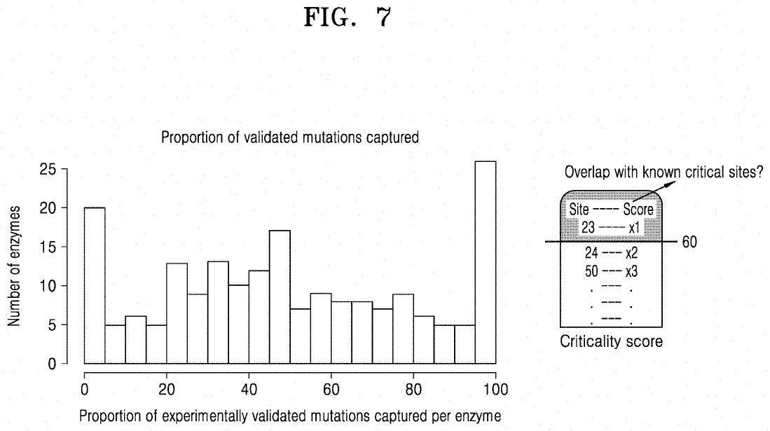

FIG. 7 is an example graph illustrating detection of critical sites for enzyme stability, according to the embodiments as disclosed herein. In 21 of the 100 enzymes in the dataset, all known sites are captured. On average, ˜60% of the known critical sites captured are in the top 40 percentile as shown in the FIG. 7.

The embodiments disclosed herein can be implemented using at least one software program running on at least one hardware device and performing network management functions to control the elements.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

What is claimed is:

1. A method for handling an enzyme function through at least one change at multiple sites in an enzyme, comprising:
detecting, by an electronic device, one or more functionally linked site pairs from the multiple sites in the enzyme;
determining, by the electronic device, at least one criticality assessment function for the one or more functionally linked site pairs;
prioritizing, by the electronic device, the one or more functionally linked site pairs based on at least one of the criticality assessment function, a functional linkage strength, and a spatial linkage metric; and
identifying, by the electronic device, the multiple sites from the prioritized functionally linked site pairs as optimum sites for introducing a change to handle the enzyme function.

2. The method of claim 1, wherein the one or more functionally linked site pairs are detected by:
determining at least one functional site;
detecting a linkage of the at least one functional site to another site; and
detecting an information flow in the linkage of the at least one functional site to the another site.

3. The method of claim 2, wherein determining the at least one functional site comprises at least one of:
identifying at least one functional site that defines a ligand binding pocket of the enzyme; and
identifying at least one functional site that defines a sequence context of a ligand binding site identified in the binding pocket of the enzyme.

4. The method of claim 2, wherein detecting the linkage of the at least one functional site to the another site comprises:
identifying at least one site linked to functional sites using an evolutionary constraint parameter; and
removing at least one functional site pair with no information flow between the site pair.

5. The method of claim 1, wherein the criticality assessment function for the linked site and the one or more functionally linked site pairs is determined based on a protein feature parameter at a specified site and a neighborhood feature parameter.

6. The method of claim 1, further comprises enabling enhancement to the enzyme function through at least one change at multiple sites in the prioritized functionally linked site pairs.

7. The method of claim 1, wherein the electronic device is used to reduce the number of functionally linked site pairs to be evaluated for user-desired changes in the enzyme function.

8. The method of claim 1, further comprising proposing, by the electronic device, at least one change at multiple sites by combining the one or more functionally linked pairs at a user defined site.

9. The method of claim 1, wherein the criticality assessment function is computed for every non-functional site, wherein a non-functional site is ranked based on a criticality score and enhances enzyme function through changes in enzyme stability through at least one change at multiple sites.

10. The method of claim 1, wherein the multiple identified sites include a block of multiple sites which are collectively handled for the enzyme function.

11. An electronic device for handling an enzyme function through at least one change at multiple sites in an enzyme, comprising:
a memory; and
a processor, coupled with the memory, configured to:
detect one or more functionally linked site pairs from the multiple sites in the enzyme;
determine at least one criticality assessment function for the one or more functionally linked site pairs;
prioritize the one or more functionally linked site pairs based on at least one of the criticality assessment function, a functional linkage strength, and a spatial linkage metric; and
identify the multiple sites from the prioritized functionally linked site pairs as optimum sites for introducing a change to handle the enzyme function.

12. The electronic device of claim 11, wherein the one or more functionally linked site pairs are detected by:
determining at least one functional site;
detecting a linkage of the at least one functional site to another site; and
detecting an information flow in the linkage of the at least one functional site to the another site.

13. The electronic device of claim 12, wherein determining the at least one functional site comprises at least one of:
identifying at least one functional site that defines a ligand binding pocket of the enzyme; and
identifying at least one functional site that defines a sequence context of a ligand binding site identified in the binding pocket of the enzyme.

14. The electronic device of claim 12, wherein detecting the linkage of the at least one functional site to another site comprises:
identifying at least one site linked to functional sites using an evolutionary constraint parameter; and
removing at least one functional site pair with no information flow between the site pair.

15. The electronic device of claim 11, wherein the criticality assessment function for the linked site and the one or more functionally linked site pairs is determined based on a protein feature parameter at a specified site and a neighborhood feature parameter.

16. The electronic device of claim 11, wherein the processor is configured to enable enhancement to the enzyme function through at least one change at multiple sites in the prioritized functionally linked site pairs.

17. The electronic device of claim 11, wherein the processor is used to reduce the number of functionally linked site pairs to be evaluated for user-desired changes in the enzyme function.

18. The electronic device of claim 11, wherein the processor is configured to propose the at least one change at multiple sites by combining the at least one functionally linked pairs at a user defined site.

19. The electronic device of claim 11, wherein the criticality assessment function is computed for every non-functional site, wherein non-functional site is ranked based on a criticality score and enhances enzyme function through changes in enzyme stability through at least one change at multiple sites.

20. The electronic device of claim 11, wherein the multiple identified sites include a block of multiple sites which are collectively handled for a desired enzyme function.
